(19) United States
(12) Patent Application Publication
(10) Pub. No.: US 2009/0099405 A1
(43) Pub. Date: Apr. 16, 2009

(54) MONOPHASIC MULTI-COIL ARRAYS FOR TRANCRANIAL MAGNETIC STIMULATION

(75) Inventors: M. Bret Schneider, Portola Valley, CA (US); David J. Mishelevich, Playa del Rey, CA (US)

Correspondence Address: SHAY GLENN LLP, 2755 CAMPUS DRIVE, SUITE 210, SAN MATEO, CA, 94403, US

(73) Assignee: NeoStim, Inc., Redwood City, CA (US)

(21) Appl. No.: 12/185,544

(22) Filed: Aug. 4, 2008

Related U.S. Application Data
(60) Provisional application No. 60/954,018, filed on Aug. 5, 2007.

(51) Int. Cl. A61N 2/02

(52) U.S. Cl. 600/13

(57) ABSTRACT

Efficient use of multi-coil arrays for magnetic nerve stimulation depends upon coordinating the coil polarity, the pulse phase and the pulse timing. Monophasic magnetic nerve stimulators produce more precise and predictable results in the stimulation of nerves than biphasic and polyphasic machines, but are less electrically efficient, and consequently limited in terms of pulse train speed. The present invention concerns the coordination of pulse polarity, phase, timing, and strength between multiple magnetic stimulation coils. The goal is to optimize the manner in which multiple coils may be used synergistically to control the activity of underlying neural tissue.

### CROSS REFERENCE TO RELATED APPLICATIONS

This application claims priority to U.S. Provisional Patent Application Ser. No. 60/954,018, filed on Aug. 5, 2007, titled “MONOPHASIC MULTI-COIL ARRAYS FOR TRANCRANIAL MAGNETIC STIMULATION.”

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The devices and methods described herein relate generally to the use of electromagnets to stimulate the brain treatment of hypertension.

### BACKGROUND OF THE INVENTION

Concurrent use of more than one magnetic stimulation coil can be used to improve depth of stimulation within a brain, and to help control the location of a deep area of stimulation, as described in Schneider M B et al. 2004 U.S. Ser. No. 10/821,807, and Mishelevich et al. 2006 U.S. Ser. No. 11/429,504. However, presently available rTMS pulse generator units are limited in their ability to provide the optimal signal to such a coil array.

Most magnetic nerve stimulators in use today are biphasic or polyphasic, for example the Magstim Rapid2 (Magstim Ltd., Wales, UK). Electrically efficient, they are well suited to sustained, rapid pulse trains needed for producing enduring brain modulation, for example depression treatments. However, the complexity of the polyphasic waveform, meeting the complexity of the nervous system frequently yields inconsistent results. In the case of multiple coil stimulation, there is an increased chance that one waveform phase from one coil could diminish or cancel a concurrent phase from another coil

Monophasic magnetic stimulators provide more predictable neurostimulation effects, and they are well known in the art. For example, the Magstim 200² (Magstim Ltd, Wales, UK), and the MagPro X100 with MagOption by the Dantec division of Medtronic (Copenhagen, Denmark), which generates pulse shapes including biphasic, monophasic, and half-sine. Electrically inefficient, these machines are not capable of sustained, rapid pulse trains. The output of these devices is always to a single output to a coil in which all loop portions receive the same electrical waveform.

The prior art does not provide any means for coordinating pulse phase, timing polarity or strength between more than one coil.

### SUMMARY OF THE INVENTION

Described herein are methods and devices for stimulating neural structures within the brain using multi-coil arrays.

A device is described in which a biphasic or polyphasic electrical discharge from a magnetic nerve stimulation or rTMS machine is split into two separate monophasic pulses. These separate pulses are then sent to two separate coils. The method provides for the coordination of pulse polarity, phase, timing, and strength between multiple magnetic stimulation coils. The goal is to optimize the manner in which multiple coils may be used synergistically to control the activity of underlying neural tissue.

In one embodiment a biphasic electrical pulse is passed through a high-power bridge rectifier, with the two outputs of which power the positive pole of one double coil and the negative pole of the other double coil, while the remaining pole of each of two double coils are both held to ground. The electrical pulses through each of the coils is thereby monophasic, and the induced magnetic field, is at least substantially monophasic.

In an alternative embodiment, the two poles of a biphasic electrical pulse generator are passed through high-power diodes, the positive pole of one double coil and the negative pole of the other double coil, while the remaining pole of each of two double coils are both held to ground. The electrical pulses through each of the coils is thereby monophasic, and the induced magnetic field, is at least substantially monophasic.

In another alternative embodiment, a biphasic electrical pulse source has its poles connected with opposite sides of a high-power bridge rectifier. The outputs from the remaining sides of the bridge rectifier are passed to the positive and negative poles of one double coil. The electrical pulses through each of the coils is thereby monophasic, and the induced magnetic field, is at least substantially monophasic.

Pulse generation devices that produce such pulses are commercially available such as the Magstim Rapid stimulator by Magstim LTD (Wales, UKCoils that are useful within the context of the present invention are commercially available, for example the 70 mm double coil (Magstim LTD (Wales, UK).

In one embodiment, coils may be situated substantially next to one another such that their magnetic fields are directed in a substantially similar direction if the coils are arranged in the same polarity, and a substantially opposite direction if the coils are arranged with opposite polarities. In another embodiment, the coils may be situated substantially opposite one another, such that their magnetic fields are directed in a substantially opposite direction if the coils are situated with the same polarity, and substantially the same if the coils are situated in opposite polarity.

### BRIEF DESCRIPTION OF THE DRAWINGS

FIG. 1 shows a circuit diagram of a full-wave rectifier circuit applied to a two-coil array.

FIG. 2 shows a circuit diagram of a half-wave rectifier circuit applied to a two-coil array.

FIG. 3 shows a full-wave bridge rectifier powering one double coil from an array. Note the intentional absence of a smoothing capacitor.

FIG. 4A illustrates the approximate waveforms of the electrical current input and outputs, respectively associated with the circuit shown in FIG. 1

FIG. 4B illustrates the approximate waveforms of the electrical current input and outputs, respectively associated with the circuit shown in FIG. 2.

FIG. 4C illustrates the approximate waveforms of the electrical current input and output, respectively associated with the circuit shown in FIG. 2.

FIG. 5A illustrates the placement of coils next to one another so as provide a similar orientation.

FIG. 5B illustrates the placement of coils on opposing sides of a patient's head so as to provide roughly opposite orientation.

### DETAILED DESCRIPTION OF THE INVENTION

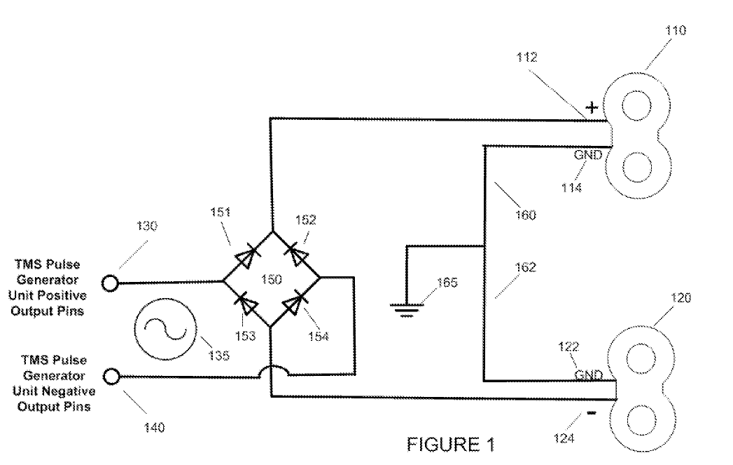

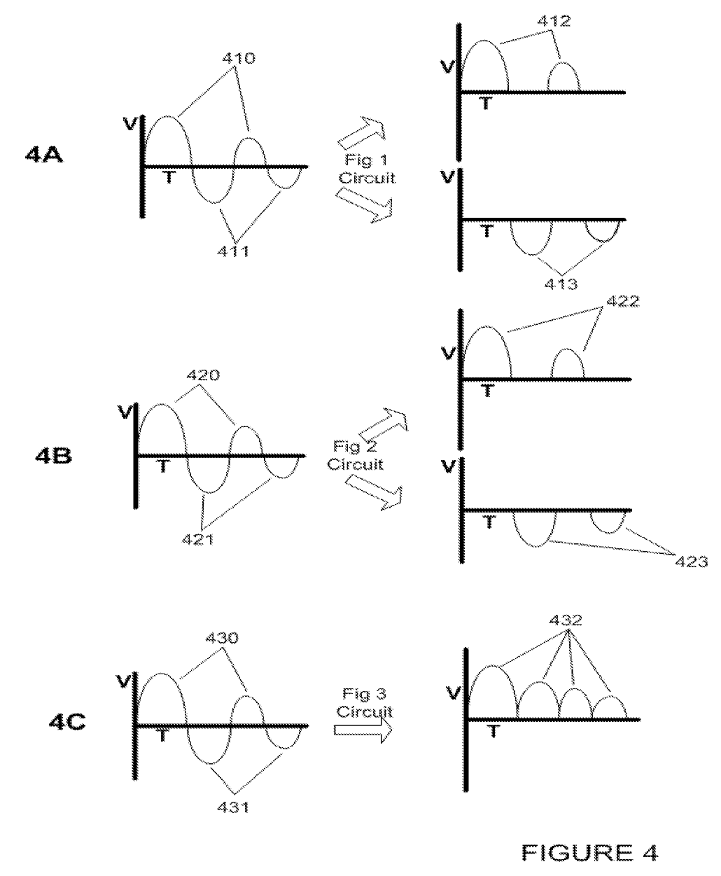

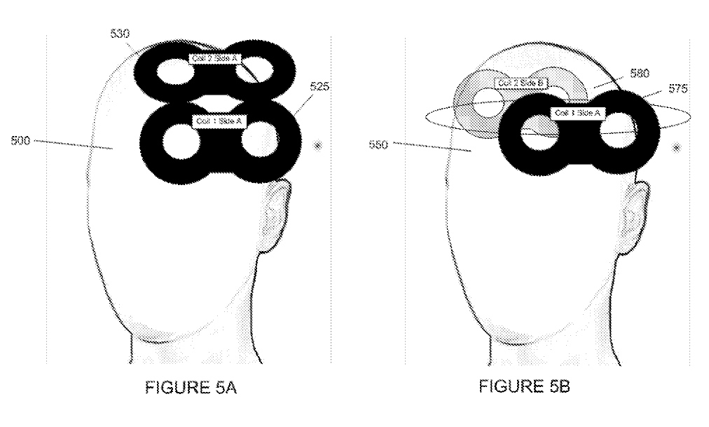

FIG. 1 shows a circuit diagram of a full-wave rectifier circuit applied to a two-coil array. In this particular embodiment, the two coils in the array, coil 110 and coil 120 are double coils, for example 70 mm double coil manufactured by Magstim Ltd. (Wales, UK). In such a double coil, two separate concentric windings are wrapped in opposite directions with a crossover between the two portions, placed such that the positive and the negative going leads to the two coil portions run electrical current in the same direction where the two portions are adjacent to one another, creating the greatest magnetic field induction under the center. Positive electrical pole 130 and negative electrical pole 140 have a pulsatile polyphasic alternating current 135 between them, and represent the black and red output wire pins on a standard repetitive transcranial magnetic stimulation device. The positive going current from pole 130 and negative going current from pole 140 enter bridge rectifier 150, which is composed of diodes 151, 152, 153 and 154. Positive portions of the waveform are then sent to positive pole 112 of TMS coil 110, while negative-going portions of the negative pole 124 of TMS coil 120. Ground pole 122 of TMS coil 120 and ground pole 114 of TMS coil 110 both go to ground 165 by leads 162, and 160, respectively. The output of this circuit will be illustrated in FIG. 4A, and positioning of these coils will be discussed with respect to FIGS. 5A and 5B. The electrical pulses through each of the coils is thereby monophasic, and the induced magnetic field, is at least substantially monophasic.

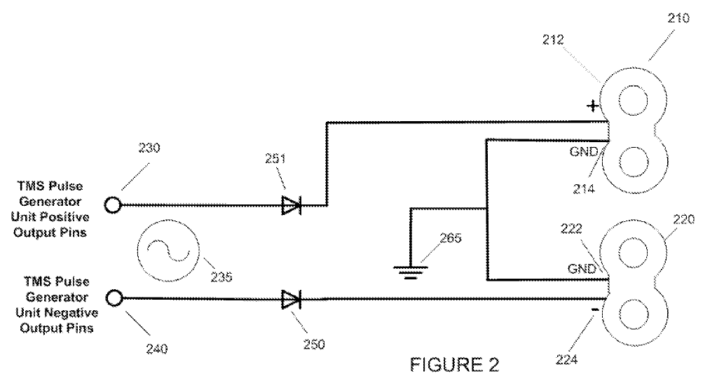

FIG. 2 shows a circuit diagram of a (half-wave) rectifier diodes applied to a two-coil array. Positive electrical pole 230 and negative electrical pole 240 have a pulsatile polyphasic alternating current 235 between them, and represent the black and red output wire pins on a standard repetitive transcranial magnetic stimulation device. The positive going current from pole 230 enters diode 251 and is passed to positive pole 212 of TMS coil 210. Meanwhile, the negative-going current from pole 240 enters diode 250 and is passed to negative pole 224 of TMS coil 220. Ground pole 222 of TMS coil 220 and ground pole 214 of TMS coil 210 both go to ground 265. The output of this circuit will be illustrated in FIG. 4B, and positioning of these coils will be discussed with respect to FIGS. 5A and 5B. The electrical pulses through each of the coils is thereby monophasic, and the induced magnetic field, is at least substantially monophasic.

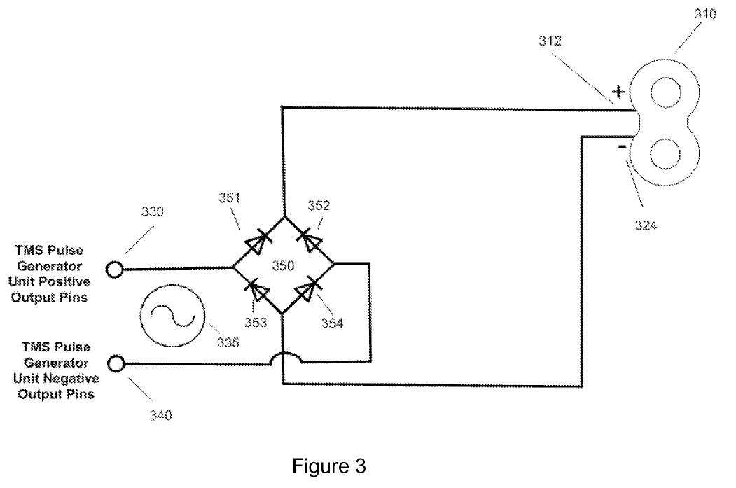

FIG. 3 illustrates the use of a full wave bridge rectifier circuit as applied to a single coil. Positive electrical pole 330 and negative electrical pole 340 have a pulsatile polyphasic alternating current 335 between them, and represent the black and red output wire pins on a standard repetitive transcranial magnetic stimulation device. The positive going current from pole 330 and negative going current from pole 340 enter bridge rectifier 350, which is composed of diodes 351, 352, 353 and 354. Positive portions the waveform are then sent to positive pole 312 of TMS coil 310, while negative-going portions of the negative pole 324 of TMS coil 310. The output of this circuit will be illustrated in FIG. 4C. The electrical pulses through the coils is thereby monophasic, and the induced magnetic field, is at least substantially monophasic.

FIG. 4A illustrates the input and outputs of the circuit shown in FIG. 1. Positive going pulses 410 and negative-going pulses 411 are input into the FIG. 1 circuit, and emerge as positive-going pulses 412 on one TMS coils, and negative-going pulses 413 on the other TMS coil. When using this configuration, the two coils may be arranged on opposite sides of the head so as to maximize summation and minimize energy cannibalization, provided that the coils are properly flipped so as to summate rather than cancel, as will be described with respect to FIGS. 5A and 5B.

FIG. 4B illustrates the input and outputs of the circuit shown in FIG. 2. Positive-going pulses 420 and negative-going pulses 421 are input into the FIG. 1 circuit, and emerge as positive-going pulses 422 on one TMS coils, and negative-going pulses 423 on the other TMS coil. When using this configuration, the two coils may also be arranged on opposite sides of the head so as to maximize summation and minimize energy cannibalization, provided that the coils are properly flipped so as to summate rather than cancel, as will be described with respect to FIGS. 5A and 5B.

FIG. 4C illustrates the input and output of the circuit shown in FIG. 3. Positive-going pulses 430 and negative-going pulses 431 are input into the FIG. 3 circuit, and emerge as rectified positive-going pulses 432 the TMS coil. In typical rectifier circuits, a capacitor is generally used to smooth the output. However, in the present invention, no capacitor is used, as unevenness, with its associated DB/DT is a desirable quality for inducing neuronal depolarization.

FIG. 5 describes two forms of spatial relationship that two or more coils in an array can have in boost the summation of their fields. FIG. 5A shows two TMS coils; coil 530 and coil 525 placed along side one another over head 500. While their positions are obviously not identical due to space that each takes up on the scalp surface, it will be appreciated that their trajectories are substantially similar. In practice this means that a significant percentage of their magnetic field output of the same phase will sum rather than cancel. FIG. 5B shows two coils; coil 575 and coil 580 facing each other from opposite sides of the patients' head 550. In practice, this position will generally cause pulses from the two coils to summate only if they are in opposite phase.

### Discussion

Using the means provided herein, biphasic electrical pulse source are divided such that the current passing through each of the coils is monophasic, and the induced magnetic field, is at least substantially monophasic. Because magnetic field is induced as a function of change in electrical current per unit time, even a perfectly monophasic electrical pulse does not create a perfectly monophasic magnetic field pulse: the pulse of electrical current cannot continue to rise indefinitely (in practice the current pulses are typically of approximately 0.1 ms in duration), and then falls to baseline. This means that there will be some reversal of induced magnetic field direction. However, in accordance with the means provided, this opposite-phase component will be of substantially less magnitude than the principal component of the magnetic field pulse.

Summation from two sources from standpoint of physics (as opposed to temporal and spatial forms of physiological summation) can be summarized as follows:
Matched phase and opposite direction yields cancellation.
Opposite phase and similar direction yields cancellation.
Matched phase and similar direction yields summation.
Opposite phase and opposite direction yields summation.

In practice under general conditions, however, side-by-side coils of same polarity and phase may either enhance or diminish the neuronal response, as the sharp DB/DT at the margin between these coils may produce a potent depolarizing effect. Likewise coils on the opposite side of the head of opposite polarity are subject to the same uncertainty. Therefore controllability of this phenomenon depends upon precise control of magnetic field direction and as well as precise controls of the coils with respect to specific anatomical geometry.

Note that phase can be changed electronically, as described herein, reversing polarity of the coil, or by simply flipping the coil face with respect to the target. In accordance with the present invention, coil polarity and direction of aim are planned in order to maximize the extent to which vectors that are desired are summated, and vectors which are not desired are cancelled.

The ability of those two coils to act synergistically to depolarize neurons, rather than to diminish each other's effects, however, depends upon depends upon coordinating the coil polarity, the pulse phase and the pulse timing, as well as the power of the sources. For example, two coils on the opposite sides of a patient's head, located 180 degrees apart, but with like coil faces (same polarity with respect to the head) may serve to diminish the stimulating effect in the area between the coils. By contrast, two side-by side magnetic coils oriented in essentially the same polarity, and pulsed simultaneously from identical sources, will summate to produce additive effects. Likewise, two coils on the opposite sides of a patient's head, located 180 degrees apart, but with one coil face flipped to provide opposite polarity, will also result in an additive effect in the space between the coils.

### REFERENCES AND PRIOR ART

Dantec magnetic stimulation product information on MagPro X100 with MagOption. http ://www.danica.nl/neuro/neuro-magnetische-stimulatoren.htm.

Magnetic Stimulation in Clinical Neurophysiology. Second Edition. Hallet M, Chokroverty S, Ed. Elsevier Inc., Philadelphia, Pa., 2005. Chapter by Ruohonen, et al. Transcranial Magnetic Stimulation: A Neurochronometrics of Mind. Walsh V, Pascual-Leone A. MIT Press. Cambridge, Mass. 2003.

Davey K, Riehl M. Deigning Designing Transcranial Magnetic Stimulation Systems. .IEEE Transactions on Magnetics. Vol. 41, No. 3, March 2005. 1142-1148.

Barker A T. An Introduction to the Basic Principles of Magnetic Nerve Stimulation. Journal of Clinical Neurophysiology. Vol 8, No. 1, 1991: 26-37.

“Robotic device for stereotactic transcranial magnetic stimulation.” Schneider M B and Mishelevich D J U.S. Ser. No. 10/821,807.

“Trajectory-Based Transcranial Magnetic Stimulation,” Mishelevich D J and Schneider M B, Pending U.S. patent application Ser. No. 11/429,504.

What is claimed is:

1. An arrangement of two or more therapeutic magnetic coils in series, in which the positive and negative poles are passed through the a bridge rectifier, whereby the electrical currents in each of the coils are substantially in single directions.

2. An arrangement of two or more therapeutic magnetic coils, in which the phase of the electrical current passing through the coils has been matched to the polarity and position of the coils such that matched coil polarity orientation and matched B-field direction yields summation.

3. An arrangement of two or more therapeutic magnetic coils, in which the phase of the electrical current passing through the coils has been matched to the polarity and position of the coils such that opposite coil polarity orientation and opposite B-field direction yields summation.

4. An arrangement of two or more therapeutic magnetic coils, in which the phase of the electrical current passing through the coils has been matched to the polarity and position of the coils such that matched phase and opposite direction yields cancellation.

5. An arrangement of two or more therapeutic magnetic coils, in which the phase of the electrical current passing through the coils has been matched to the polarity and position of the coils such that opposite phase and matched direction yields cancellation.
